(19) United States
(12) Patent Application Publication
(10) Pub. No.: US 2024/0301027 A1
(43) Pub. Date: Sep. 12, 2024

(54) TREATMENT OF MAGE-A4 POSITIVE CANCER

(71) Applicant: Immunocore Limited, Abingdon (GB)

(72) Inventors: Mohammed DAR, Abingdon (GB); Shannon MARSHALL, Abingdon (GB); Peter Christopher HOLLAND, Abingdon (GB)

(73) Assignee: Immunocore Limited, Abingdon (GB)

(21) Appl. No.: 18/668,935

(22) Filed: May 20, 2024

Related U.S. Application Data
(63) Continuation of application No. PCT/EP2022/083928, filed on Nov. 30, 2022.
(60) Provisional application No. 63/285,035, filed on Dec. 1, 2021.

(51) Int. Cl. C07K 14/725; A61K 9/00; A61K 38/00; A61K 45/06; A61P 35/00

(52) U.S. Cl. CPC C07K 14/7051; A61K 9/0019; A61K 45/06; A61P 35/00; A61K 38/00; C07K 2317/565; C07K 2317/622; C07K 2319/30

(57) ABSTRACT

Methods are presented for the administration of a TCR-anti-CD3 fusion molecule to treat patients who have a MAGE-A4 positive cancer. The methods comprise administering an TCR-anti-CD3 fusion molecule to a patient intravenously and comprise administration of (a) at least one first dose in the range of from 10-20 μg; (b) at least one second dose in the range of from 40-50 μg; and then (c) at least one third dose in the range of from 90-400 μg, wherein doses are administered every 6-8 days.

### CROSS REFERENCE TO RELATED APPLICATIONS

This application claims the priority benefit of U.S. Provisional Application Ser. No. 63/285,035, filed Dec. 1, 2021, which is hereby incorporated by reference in its entirety.

### REFERENCE TO AN ELECTRONIC SEQUENCE LISTING

The contents of the electronic sequence listing (146392060040SEQLIST.xml; Size: 28,393 bytes; and Date of Creation: Nov. 23, 2022) are incorporated herein by reference in their entirety.

### FIELD

The present invention relates to the treatment of cancer, particularly MAGE-A4 positive cancers. In particular, it relates to a dosage regimen for a T cell redirecting bispecific therapeutic (TCR-anti-CD3 fusion molecule) comprising a T cell receptor (TCR) that binds the HLA-A*02 restricted peptide GVYDGREHTV (SEQ ID NO: 1), fused to an anti-CD3 scFv.

### BACKGROUND

MAGE-A4 belongs to the MAGE family of germline encoded cancer antigens (De Plaen, et al., (1994), Immunogenetics 40(5): 360-369) and has the Uniprot accession number P43358. Such antigens have been found to be frequently expressed in a variety of cancers, while their expression in normal tissues is limited to adult testes and other immune-privileged sites including placenta. The cancer specific nature of these genes makes them ideal targets for anti-cancer therapeutics. The precise function of MAGE-A4 remains unknown but it is believed to play a role in embryonic development. High level expression of MAGE-A4 has been reported in tumours of several types including melanoma, carcinomas of the esophagus, the head and neck, the lung, the breast and the bladder (Bergeron, (2009), Int J Cancer 125(6): 1365-1371; Cabezon, et al., (2013), Mol Cell Proteomics 12(2): 381-394; Cuffel, et al., (2011), Int J Cancer 128(11): 2625-2634; Forghanifard, et al., (2011), Cancer Biol Ther 12(3): 191-197; Karimi, et al., (2012), Clin Lung Cancer 13(3): 214-219; Svobodova, et al., (2011), Eur J Cancer 47(3): 460-469). The 10-mer peptide GVYDGREHTV (SEQ ID NO: 1) corresponds to amino acids 230-239 of the full length MAGE-A4 protein. This peptide binds to HLA-A*02 and the peptide-HLA complex has been shown to stimulate cytotoxic T cells leading to lysis of MAGE-A4 positive, HLA-A*02 positive, tumour cells (Duffour, et al., (1999), Eur J Immunol 29(10): 3329-3337 and WO2000020445). The GVYDGREHTV HLA-A*02 complex therefore provides a useful target antigen for immunotherapeutic intervention.

WO2017/175006 describes TCRs that bind to the GVYDGREHTV HLA-A*02 complex. The TCRs are mutated relative to a native MAGE-A4 TCR alpha and/or beta variable domains to have improved binding affinities for, and/or binding half-lives for the complex, and can be associated (covalently or otherwise) with a therapeutic agent. One such therapeutic agent is an anti-CD3 antibody, or a functional fragment or variant of said anti-CD3 antibody such as a single chain variable fragment (scFv). The anti-CD3 antibody or fragment may be covalently linked to the C- or N-terminus of the alpha or beta chain of the TCR. The resulting molecule is a TCR bispecific. TCR bispecific molecules that target MAGEA4 are also described in WO2021023658.

TCR bispecific proteins redirect polyclonal T cells to target peptides derived from intra- or extra-cellular disease associated antigens and presented on the cell surface in complex with an HLA molecule. This approach has been tested clinically in the context of a different antigen with a TCR bispecific protein targeting a HLA-A*02 restricted peptide from gp 100 and CD3(tebentafusp). Administration of this molecule provided an OS benefit in uveal melanoma (Nathan P, et al. Overall Survival Benefit with Tebentafusp in Metastatic Uveal Melanoma. N Engl J Med 2021: 385:1196-1206). However, no such TCR bispecific proteins targeting MAGE-A4 have been tested clinically.

IMC-C103C is a T cell redirecting bispecific therapeutic agent comprising a soluble affinity enhanced TCR that binds to the GVYDGREHTV peptide—HLA-A*02 complex, fused to an anti-CD3 scFv. The targeting end of IMC-C103C (the soluble TCR) binds to a peptide fragment of the MAGE-A4 tumor antigen presented by HLA-A*02 on the surface of cancer cells. HLA molecules are polymorphic; approximately 47% of Caucasian individuals in the US and European countries express the HLA-A*02 genotype with the HLA-A*02:01 allele detected in more than 95% of HLA-A*02-positive individuals. The effector end of IMC-C103C (anti-CD3 scFv) can bind to CD3 on any T cell, redirecting the T cell to produce effector cytokines and/or kill the cell presenting the target. In addition, IMC-C103C-mediated tumor lysis may prime an endogenous anti-tumor immune response. ImmTAC such as IMC-C103C are highly potent molecules, with redirection of T-cell activity observed against tumor cell lines presenting as few as 10 to 50 target peptide:HLA complexes. IMC-C103C has been shown to selectively redirect T cell activity in the presence of HLA-A*02:01-positive/MAGE-A4-positive cell lines, leading to T cell activation and killing of MAGE-A4-positive cancer cells, at concentrations as low as 1 pM to 10 pM. As described above, the HLA-A*02 restricted peptide GVYDGREHTV (SEQ ID NO: 1) is derived from the germline cancer antigen MAGE-A4. IMC-C103C has a TCR alpha chain amino acid sequence of SEQ ID NO: 14 and a TCR beta chain-anti-CD3 amino acid sequence of SEQ ID NO: 16.

The safety and tolerability of IMC-C103C in patients with selected advanced solid tumours is being investigated in a Phase I trial (clinical trial identifier NCT03973333).

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In a first aspect, the present invention provides a TCR-anti-CD3 fusion molecule comprising:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating MAGE-A4 positive cancer in a patient comprising administering the TCR-anti-CD3 fusion molecule to said patient intravenously, wherein the method comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days.

In another aspect, the present invention provides a method of treating MAGE-A4 positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days.

In some embodiments, the TCR-anti-CD3 fusion molecule comprises an alpha chain amino acid sequence corresponding to SEQ ID NO: 14 and a TCR beta chain-anti-CD3 amino acid sequence corresponding to SEQ ID NO: 16. In some embodiments, the first dose is 15 μg, the second dose is 45 μg and the third dose is in the range of from 140 μg-240 μg. In some embodiments, the third dose is 140 μg, 180 μg or 240 μg. In some embodiments, the first dose is 15 μg, the second dose is 45 μg, and the third dose is 90 μg, 140 μg, 180 μg, or 240 μg. In some embodiments, a further third dose is administered every 6-8 days until treatment is stopped. In some embodiments, a steroid is administered prior to the first, second and/or third dose. In some embodiments, the TCR-anti-CD3 fusion molecule is administered in combination with one or more anti-cancer therapies. In some embodiments, the anti-cancer therapy is a checkpoint inhibitor. In some embodiments, the checkpoint inhibitor is atezolizumab. In some embodiments, the MAGE-A4 positive cancer is selected from the group consisting of ovarian cancer, lung cancer, head and neck cancer, oesophageal cancer, breast cancer, synovial sarcoma, gastric cancer, bladder cancer and a tumour with squamous cell histology.

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIG. 1 provides the amino acid sequences referred to in the present application.

FIG. 2 shows the design of the study described in the Example.

FIG. 3 shows the dose escalation schema for the study described in the Example.

FIG. 4 shows the results of weekly IV dosing with 20 hr half-life in peripheral blood.

FIG. 5 shows consistent and robust evidence of T cell activity at ≥90 mcg IMC-C103C. Concentrations <lower limit of detection (LLOD) were set to half LLOD for purposes of deriving fold change. Fold increase compares pre-dose to maximum post-dose (4 hr, 8 hr, and 24 hr timepoints). 24 patients were evaluable (pre and post-dose cytokine results available for the Day 15 dose). *Day 16 ALC was only analyzed following introduction of intra-patient dose escalation; therefore, not collected in first cohorts.

FIG. 6 shows cytokine induction associated with tumor MAGE-A4 expression. Concentrations <LLOD were set to half LLOD for purposes of deriving fold change. Fold increase compares pre-dose to maximum post-dose (4 hr, 8 hr, and 24 hr timepoints). 29 patients were evaluable (15 mcg on Day 1, pre and post-dose cytokine results and MAGE-A4 results available).

FIGS. 7A & 7B show increased T cell infiltration into MAGE-A4+ tumors. FIG. 7A shows infiltration of CD3+ T cells; FIG. 7B shows infiltration of CD8+ T cells.

FIGS. 8A & 8B show clinical activity in ovarian carcinoma at doses of 90-240 mcg. FIG. 8A shows best % change in tumor size from baseline. FIG. 8B shows % change in tumor size from baseline over time.

FIG. 9 shows overall survival in patients with ovarian carcinoma at doses ≥15 mcg.

### DETAILED DESCRIPTION

It is to be understood that this disclosure is not limited to particular compositions or biological systems, which can, of course, vary. It is also to be understood that the terminology used herein is for the purpose of describing particular embodiments only, and is not intended to be limiting.

In a first aspect, the present invention provides a TCR-anti-CD3 fusion molecule comprising:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating MAGE-A4 positive cancer in a patient comprising administering the TCR-anti-CD3 fusion molecule to said patient intravenously, wherein the method comprises administration of:
(a) at least one first dose in the range of from 10-20 μg;
(b) at least one second dose in the range of from 40-50 μg; and then
(c) at least one third dose in the range of from 90-400 μg,
   wherein doses are administered every 6-8 days.

Several risk factors may be associated with the administration of TCR bispecific reagents, including cytokine release syndrome (CRS), local tumour inflammation, cytopenia and off target T cell activation. In some cases, dose limiting toxicities may arise at doses below a clinically effective dose. Furthermore, higher doses may result in off target recognition of normal tissues. The inventors have surprisingly found an intra-patient dose escalation regimen that allows IMCC103C to be administered with a manageable safety profile and that demonstrates clinical activity.

The TCR-anti-CD3 fusion molecule for use in the invention comprises an anti-CD3 scFv covalently linked to the N-terminus of the beta chain of a TCR via a linker. This type of molecule is known as an ImmTAC® (Immune Mobilizing Monoclonal TCRs Against Cancer). ImmTAC® molecules are engineered to activate a potent T cell response to specifically kill target cancer cells. TCR-anti-CD3 fusion molecules for use in the invention (i.e. ImmTACs targeting MAGE-A4) are described in WO2017/175006, which is incorporated by reference herein in its entirety.

The terms “TCR-anti-CD3 fusion molecule”, “ImmTAC” and “T cell redirecting bispecific therapeutic agent” are used interchangeably herein.

The term “TCR beta chain-anti-CD3” used herein refers to the TCR beta chain portion of the ImmTAC together with the linker and the anti-CD3 scFv. The term “beta chain” is sometimes used in relation to ImmTACs as an alternative way to describe this portion of the molecule.

In some embodiments, the TCR-anti-CD3 fusion molecule for use in the invention comprises:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

In other words, although the molecule may have some variation in the TCR alpha chain amino acid sequence compared to the sequence of SEQ ID NO: 14 (as long as the TCR alpha chain amino acid sequence has at least 90% identity to SEQ ID NO: 14) and/or some variation in the TCR beta chain-anti-CD3 amino acid sequence compared to the sequence of SEQ ID NO: 16 (as long as the TCR beta chain-anti-CD3 amino acid sequence has at least 90% identity to the amino acid sequence of SEQ ID NO: 16), the CDRs of the TCR alpha chain must have the amino acid sequences of SEQ ID NOs: 3, 4 and 5 respectively and the CDRs of TCR beta chain must have the amino acid sequences of SEQ ID NOs: 9, 10 and 11 respectively. The requirement for the TCR alpha chain variable domain to comprise CDRs 1, 2 and 3 having the amino acid sequences of SEQ ID NOs: 3, 4 and 5 respectively and the requirement for the TCR beta chain variable domain to comprise CDRs 1, 2 and 3 having the amino acid sequences of SEQ ID NOs: 9, 10 and 11 respectively thus applies to all aspects and embodiments of the invention described herein. The TCR alpha chain variable domain thus comprises CDRs 1, 2 and 3 having 100% identity to the amino acid sequences of SEQ ID NOs: 3, 4 and 5 respectively and the TCR beta chain variable domain comprises CDRs 1, 2 and 3 having 100% identity to the amino acid sequences of SEQ ID NOs: 9, 10 and 11 respectively.

Within the scope of the invention are phenotypically silent variants of the TCR-anti-CD3 fusion molecule designated as IMC-C103C, which has a TCR alpha chain amino acid sequence corresponding to SEQ ID NO: 14 and a TCR beta chain-anti-CD3 amino acid sequence corresponding to SEQ ID NO: 16. As used herein the term “phenotypically silent variant” is understood to refer to a TCR-anti-CD3 fusion molecule which incorporates one or more further amino acid changes, including substitutions, insertions and deletions, compared to the sequences of SEQ ID NO: 14 and SEQ ID NO: 16 and which TCR-anti-CD3 fusion molecule has a similar phenotype to or the same phenotype as the TCR-anti-CD3 fusion molecule designated as IMC-C103C. For the purposes of this application, TCR-anti-CD3 fusion molecule phenotype comprises antigen binding affinity (K_{D} and/or binding half-life) and antigen specificity. A phenotypically silent variant may have a K_{D} and/or binding half-life for the GVYDGREHTV (SEQ ID NO: 1) HLA-A*02 complex within 50%, or more preferably within 20%, of the measured K_{D} and/or binding half-life of the TCR-anti-CD3 fusion molecule designated as IMC-C103C, when measured under identical conditions (for example at 25° C. and/or on the same SPR chip). Suitable conditions are further provided in Example 3 of WO2017/175006, which is incorporated herein by reference. Antigen specificity is further defined below. As is known to those skilled in the art, it may be possible to produce TCRs that incorporate changes in the variable domains thereof compared to those detailed above without altering the affinity of the interaction with the GVYDGREHTV (SEQ ID NO: 1) HLA-A*02 complex. In particular, such silent mutations may be incorporated within parts of the sequence that are known not to be directly involved in antigen binding. Such trivial variants are included in the scope of this invention.

Phenotypically silent variants may contain one or more conservative substitutions and/or one or more tolerated substitutions. Tolerated and conservative substitutions may result in a change in the K_{D} and/or binding half-life for the GVYDGREHTV (SEQ ID NO: 1) HLA-A*02 complex within 50%, or more preferably within 20%, even more preferably within 10%, of the measured K_{D} and/or binding half-life of the TCR-anti-CD3 fusion molecule designated as IMC-C103C, when measured under identical conditions (for example at 25° C. and/or the same SPR chip), provided that the change in K_{D} does not result in the affinity being less than (i.e. weaker than) 200 μm. By tolerated substitutions it is meant those substitutions which do not fall under the definition of conservative as provided below but are nonetheless phenotypically silent.

The TCR-anti-CD3 fusion molecule for use in the present invention may include one or more conservative substitutions which have a similar amino acid sequence and/or which retain the same function (i.e. are phenotypically silent as defined above). The skilled person is aware that various amino acids have similar properties and thus substitutions between them are “conservative”. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a TCR-anti-CD3 fusion molecule comprising an amino acid sequence described above but with one or more conservative substitutions and/or one or more tolerated substitutions in the sequence, such that the TCR alpha chain amino acid sequence has at least 90% identity (such as 91%, 92%, 93%, 94%, 95%, 96%, 97%, 98% or 99% identity) to the amino acid sequence of SEQ ID NO: 14, and the TCR beta chain-anti-CD3 amino acid sequence has at least 90% identity (such as 91%, 92%, 93%, 94%, 95%, 96%, 97%, 98% or 99% identity) to the amino acid sequence of SEQ ID NO: 16, and provided that the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules. BLAST protein searches can be performed with the XBLAST program, score=50, wordlength=3 to obtain amino acid sequences homologous to protein molecules for use in the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Mutations, including conservation and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6. The TCR sequences provided by the invention may be obtained from solid state synthesis, or any other appropriate method known in the art.

The TCR-anti-CD3 fusion molecules for use in the present invention have the property of binding the GVYDGREHTV (SEQ ID NO: 1) HLA-A*02 complex. TCR-anti-CD3 fusion molecules for use in the present invention have been found to strongly recognise this epitope relative to other, irrelevant epitopes, and are thus particularly suitable as targeting vectors for delivery of therapeutic agents or detectable labels to cells and tissues displaying those epitopes. Specificity in the context of TCR-anti-CD3 fusion molecule for use in the present invention relates to their ability to recognise HLA-A*02 target cells that are antigen positive, whilst having minimal ability to recognise HLA-A*02 target cells that are antigen negative.

Specificity can be measured in vitro, for example in cellular assays such as those described in Example 6 of WO2017/175006, which is incorporated herein by reference. Recognition may be determined by measuring the level of T cell activation in the presence of a TCR-anti-CD3 fusion molecule for use in the invention and target cells. Minimal recognition of antigen negative target cells is defined as a level of T cell activation of less than 20%, preferably less than 10%, preferably less than 5%, and more preferably less than 1%, of the level produced in the presence of antigen positive target cells, when measured under the same conditions and at a therapeutically relevant concentration. For TCR-anti-CD3 fusion molecules for use in the invention, a therapeutically relevant concentration may be defined as below 10 nM, for example below 1 nM or below 100 pM. Antigen positive cells may be obtained by peptide-pulsing using a suitable peptide concentration to obtain a level of antigen presentation comparable to cancer cells (for example, 10⁻⁹ M peptide, as described in Bossi et al., (2013) Oncoimmunol. 1;2 (11):e26840) or, they may naturally present said peptide. Preferably, both antigen positive and antigen negative cells are human cells. Preferably antigen positive cells are human cancer cells. Antigen negative cells preferably include those derived from healthy human tissues.

Specificity may additionally, or alternatively, relate to the ability of a TCR-anti-CD3 fusion molecule to bind to GVYDGREHTV (SEQ ID NO: 1) HLA-A*02 complex and not to a panel of alternative peptide-HLA complexes. This may, for example, be determined by the Biacore method of Example 3 of WO2017/175006, which is incorporated herein by reference. Said panel may contain at least 5, and preferably at least 10, alternative peptide-HLA-A*02 complexes. The alternative peptides may share a low level of sequence identity with SEQ ID NO: 1 and may be naturally presented. Alternative peptides may be derived from proteins expressed in healthy human tissues. Binding to GVYDGREHTV-HLA-A*02 complex may be at least 2 fold greater than to other naturally-presented peptide HLA complexes, more preferably at least 10 fold, or at least 50 fold or at least 100 fold greater, even more preferably at least 400 fold greater.

An alternative or additional approach to determine TCR specificity may be to identify the peptide recognition motif of the TCR using sequential mutagenesis, e.g. alanine scanning. Residues that form part of the binding motif are those that are not permissible to substitution. Non permissible substitutions may be defined as those peptide positions in which the binding affinity of the TCR is reduced by at least 50%, or preferably at least 80% relative to the binding affinity for the non-mutated peptide. Such an approach is further described in Cameron et al., (2013), Sci Transl Med. 2013 Aug. 7; 5 (197): 197ra103 and WO2014096803. TCR specificity in this case may be determined by identifying alternative motif containing peptides, particularly alternative motif containing peptides in the human proteome, and testing these peptides for binding to the TCR. Binding of the TCR to one or more alternative peptides may indicate a lack of specificity. In this case further testing of TCR specificity via cellular assays may be required.

As is known to those skilled in the art, peptides derived from MAGE family members may share a high level of sequence identity with peptides derived from other MAGE family members. For example, there are peptides derived from MAGE-A8 and MAGE-B2 that differ by only two residues from SEQ ID NO 1 (GVYDGREHTV). Said peptides and cells expressing said MAGE family members may be excluded from the definition of specificity provided above, particularly if said MAGE family members are known to be cancer antigens, such as MAGE-A8 and MAGE-B2. TCR-anti-CD3 fusion molecules for use in the invention may therefore recognise peptides with high percentage sequence identity that are derived from other MAGE family members, including MAGE-A8 and MAGE-B2 and displayed in the context of HLA A*02. Recognition of said peptides by TCRs of the invention may be at a similar or lower level than recognition of GVYDGREHTV (SEQ ID NO: 1) HLA-A*02.

Preferred embodiments of TCR anti-CD3 fusion molecules for use in the invention comprise a TCR alpha chain amino acid sequence that has at least 91%, 92%, 93%, 94%, 95%, 96%, 97%, 98% or 99% identity to the amino acid sequence set forth in SEQ ID NO: 14, and a TCR beta chain-anti-CD3 amino acid sequence that has at least 91%, 92%, 93%, 94%, 95%, 96%, 97%, 98% or 99% identity, to the amino acid sequence set forth in SEQ ID NO: 16, as long as the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively. The TCR anti-CD3 fusion molecules for use in the invention may therefore vary in any region other than the CDRs.

For example, a TCR anti-CD3 fusion molecule for use in the invention may comprise a TCR alpha chain Framework 1 region (FR1) amino acid sequence that has at least 91%, 92%, 93%, 94%, 95%, 96%, 97%, 98%, 99% or 100% identity to the amino acid sequence set forth in SEQ ID NO: 27, and/or a TCR alpha chain Framework 2 region (FR2) amino acid sequence that has at least 91%, 92%, 93%, 94%, 95%, 96%, 97%, 98%, 99% or 100% identity to the amino acid sequence set forth in SEQ ID NO: 6, and/or a TCR alpha chain Framework 3 region (FR3) amino acid sequence that has at least 91%, 92%, 93%, 94%, 95%, 96%, 97%, 98%, 99% or 100% identity, to the amino acid sequence set forth in SEQ ID NO: 7 and/or a TCR alpha chain Framework 4 region (FR4) amino acid sequence that has at least 91%, 92%, 93%, 94%, 95%, 96%, 97%, 98%, 99% or 100% identity, to the amino acid sequence set forth in SEQ ID NO: 28. For example, the alpha chain FR1 and/or FR2 and/or FR3 and/or FR4 regions may each contain one or more, for example one, two or three, conservative substitutions and/or up to three tolerated substitutions.

A TCR anti-CD3 fusion molecule for use in the invention may alternatively or additionally comprise a TCR beta chain Framework 1 region (FR1) amino acid sequence that has at least 91%, 92%, 93%, 94%, 95%, 96%, 97%, 98%, 99% or 100% identity to the amino acid sequence set forth in SEQ ID NO: 29, and/or a TCR beta chain Framework 2 region (FR2) amino acid sequence that has at least 91%, 92%, 93%, 94%, 95%, 96%, 97%, 98%, 99% or 100% identity to the amino acid sequence set forth in SEQ ID NO: 12, and/or a TCR beta chain Framework 3 region (FR3) amino acid sequence that has at least 91%, 92%, 93%, 94%, 95%, 96%, 97%, 98%, 99% or 100% identity, to the amino acid sequence set forth in SEQ ID NO: 13 and/or a TCR beta chain Framework 4 region (FR4) amino acid sequence that has at least 91%, 92%, 93%, 94%, 95%, 96%, 97%, 98%, 99% or 100% identity, to the amino acid sequence set forth in SEQ ID NO: 30. For example, the beta chain FR1 and/or FR2 and/or FR3 and/or FR4 regions may each contain one or more, for example one, two or three, conservative substitutions and/or up to three tolerated substitutions.

A TCR anti-CD3 fusion molecule for use in the invention may alternatively or additionally comprise a TCR alpha chain variable domain amino acid sequence that has at least 91%, 92%, 93%, 94%, 95%, 96%, 97%, 98%, 99% or 100% identity to the amino acid sequence set forth in SEQ ID NO: 2, and/or a TCR beta chain variable domain amino acid sequence that has at least 91%, 92%, 93%, 94%, 95%, 96%, 97%, 98%, 99% or 100% identity, to the amino acid sequence set forth in SEQ ID NO: 8. For example, the TCR-anti-CD3 fusion molecule may comprise a TCR alpha chain variable domain having the amino acid sequence of SEQ ID NO: 2 and a TCR beta chain variable domain having the amino acid sequence of SEQ ID NO: 8. Alternatively, the TCR-anti-CD3 fusion molecule may comprise a TCR alpha chain variable domain having one or more mutations, for example one, two or three, conservative substitutions and/or up to three tolerated substitutions, compared to the amino acid sequence of SEQ ID NO: 2 and/or a TCR beta chain variable domain having one or more mutations, for example one, two or three, conservative substitutions and/or up to three tolerated substitutions, compared to the amino acid sequence of SEQ ID NO: 8.

A TCR anti-CD3 fusion molecule for use in the invention may alternatively or additionally comprise a TCR alpha chain constant region amino acid sequence that has at least 91%, 92%, 93%, 94%, 95%, 96%, 97%, 98%, 99% or 100% identity to the amino acid sequence set forth in SEQ ID NO: 15, and/or a TCR beta chain constant region amino acid sequence that has at least 91%, 92%, 93%, 94%, 95%, 96%, 97%, 98%, 99% or 100% identity, to the amino acid sequence set forth in SEQ ID NO: 19. For example, the TCR alpha chain constant region may have one, two or three, conservative substitutions and/or up to three tolerated substitutions, compared to the amino acid sequence of SEQ ID NO: 15. For example, the TCR beta chain constant region may have one, two or three, conservative substitutions and/or up to three tolerated substitutions, compared to the amino acid sequence of SEQ ID NO: 19.

Alternatively or additionally, the anti-CD3 scFv in the TCR anti-CD3 fusion molecule for use in the invention may comprise an amino acid sequence that has at least 91%, 92%, 93%, 94%, 95%, 96%, 97%, 98%, 99% or 100% identity to the amino acid sequence set forth in SEQ ID NO: 17.

In the TCR anti-CD3 fusion molecule for use in the invention, the TCR beta chain is linked to the anti-CD3 antibody sequence via a linker. The amino acid sequence of the linker may be selected from the group consisting of GGGGS (SEQ ID NO: 18), GGGSG (SEQ ID NO: 20), GGSGG (SEQ ID NO: 21), GSGGG (SEQ ID NO: 22), GSGGGP (SEQ ID NO: 23), GGEPS (SEQ ID NO: 24), GGEGGGP (SEQ ID NO: 25), and GGEGGGSEGGGS (SEQ ID NO: 26). Typically, the linker sequence is GGGGS (SEQ ID NO: 18). Alternatively, the linker may have one or more mutations, for example one, two or three, conservative substitutions and/or up to three tolerated substitutions compared to any of the linker sequences of SEQ ID NOs: 18 and 20-26.

It can therefore be seen that any of these embodiments may be combined as long as the resulting TCR-anti-CD3 fusion molecule comprises a TCR alpha chain amino acid sequence that has at least 90% identity (for example at least 91%, 92%, 93%, 94%, 95%, 96%, 97%, 98%, 99% or 100% identity) to the amino acid sequence of SEQ ID NO: 14, and a TCR beta chain-anti-CD3 amino acid sequence that has at least 90% identity (for example at least 91%, 92%, 93%, 94%, 95%, 96%, 97%, 98%, 99% or 100% identity) to the amino acid sequence of SEQ ID NO: 16, and the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

The TCR-anti-CD3 fusion molecule for use in the invention may comprise a TCR alpha chain having an amino acid sequence corresponding to SEQ ID NO: 14 and a TCR beta chain-anti-CD3 amino acid sequence corresponding to SEQ ID NO: 16. These are the TCR alpha chain amino acid sequence and the TCR beta chain-anti-CD3 amino acid sequence respectively of IMC-C103C.

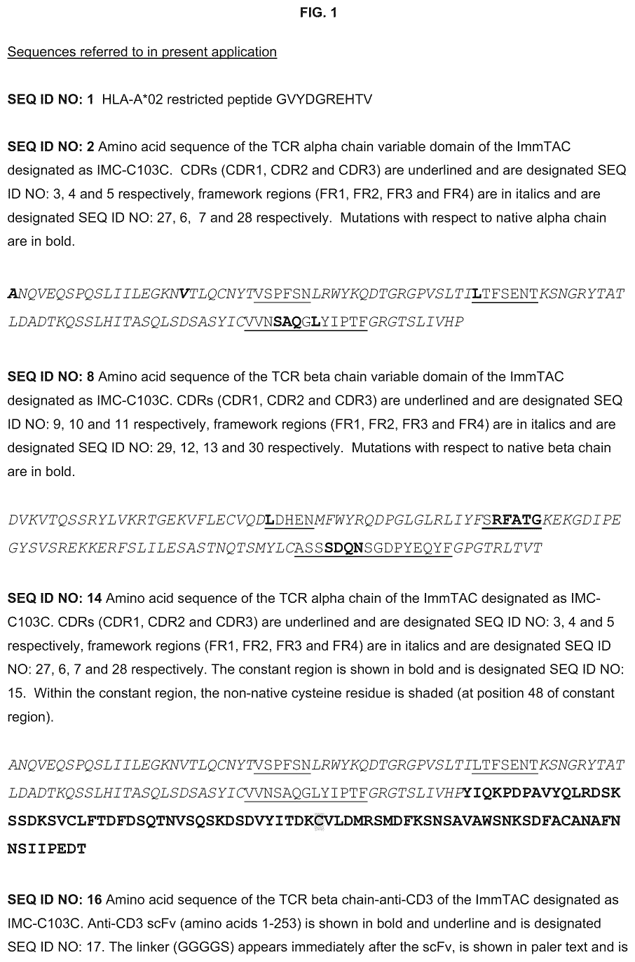

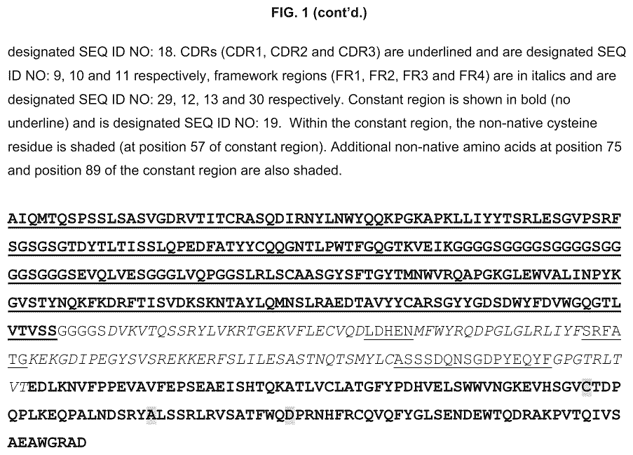

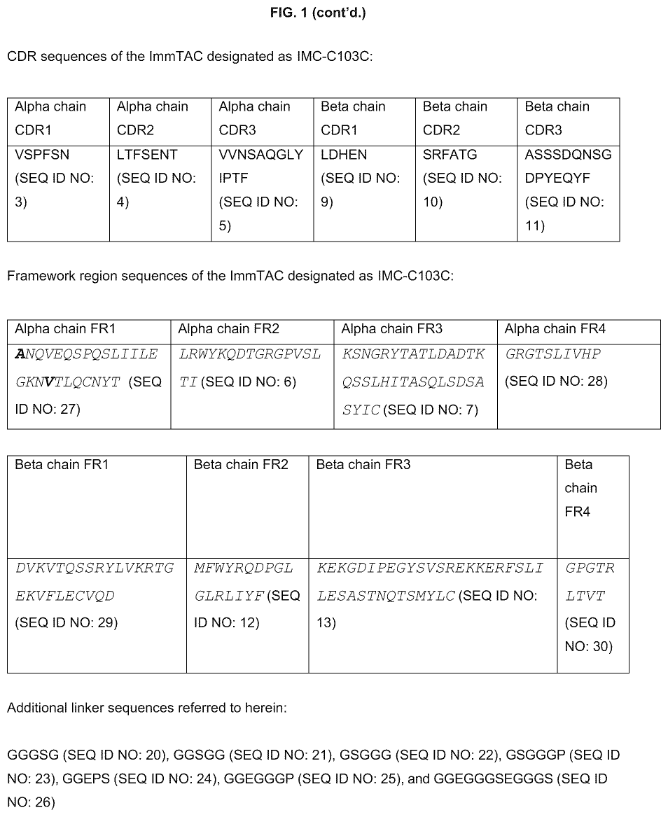

The alpha chain constant region having the amino acid sequence of SEQ ID NO: 15 includes a modification relative to the corresponding native/naturally occurring alpha chain whereby amino acid T48 of the constant region is replaced with C48, as shown in FIG. 1. The beta chain constant region having the amino acid sequence of SEQ ID NO: 19 also includes a modification relative to the native/naturally occurring beta chain whereby S57 is replaced with C57, as shown in FIG. 1. These cysteine substitutions relative to the native alpha and beta chain constant chain sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage (Li et al., Nat Biotechnol 2005 March; 23(3):349-54). In addition, the use of the stable disulphide linked soluble TCR enables more convenient assessment of binding affinity and binding half-life. The beta chain constant region having the amino acid sequence of SEQ ID NO: 19 also includes additional non-native amino acids at positions 75 (A75) and 89 (D89), as shown in FIG. 1.

All of the sequences referred to in the present application are also referred to in WO 2017/175006, which is incorporated by reference herein. Table 1 shows how the parts of the ImmTAC molecule and the SEQ ID NOs referred to herein correspond to the SEQ ID NOs of WO 2017/175006.

TABLE 1

| | | SEQ ID NO | |
|---|---|---|---|
| | SEQ ID NO | referred to in | |
| Description | referred to | WO 2017/ | Additional |
| of sequence | herein | 175006 | notes |
| HLA-A*02 restricted | SEQ ID NO: 1 | SEQ ID NO: 1 | |
| peptide derived from | | | |
| germline cancer antigen | | | |
| MAGE-A4 | | | |
| TCR alpha chain | SEQ ID NO: 2 | SEQ ID NO: 24 | Referred to as |
| variable domain of the | | | a13kaLQ |
| ImmTAC designated | | | in WO 2017/ |
| as IMC-C103C | | | 175006 |
| TCR alpha chain CDR1 | SEQ ID NO: 3 | — | |
| of IMC-C103C | | | |
| TCR alpha chain CDR2 | SEQ ID NO: 4 | — | |
| of IMC-C103C | | | |
| TCR alpha chain CDR3 | SEQ ID NO: 5 | — | |
| of IMC-C103C | | | |
| TCR alpha chain FR1 | SEQ ID NO: 27 | | |
| of IMC-C103C | | | |
| TCR alpha chain FR2 | SEQ ID NO: 6 | — | |
| of IMC-C103C | | | |
| TCR alpha chain FR3 | SEQ ID NO: 7 | — | |
| of IMC-C103C | | | |
| TCR alpha chain FR4 | SEQ ID NO: 28 | | |
| of IMC-C103C | | | |
| TCR beta chain variable | SEQ ID NO: 8 | SEQ ID NO: 29 | Referred to as |
| domain of the ImmTAC | | | b21L in WO |
| designated as IMC- | | | 2017/175006 |
| C103C | | | |
| TCR beta chain CDR1 | SEQ ID NO: 9 | — | |
| of IMC-C103C | | | |
| TCR beta chain CDR2 | SEQ ID NO: 10 | — | |
| of IMC-C103C | | | |
| TCR beta chain CDR3 | SEQ ID NO: 11 | — | |
| of IMC-C103C | | | |
| TCR beta chain FR1 of | SEQ ID NO: 29 | | |
| IMC-C103C | | | |
| TCR beta chain FR2 of | SEQ ID NO: 12 | — | |
| IMC-C103C | | | |
| TCR beta chain FR3 of | SEQ ID NO: 13 | — | |
| IMC-C103C | | | |
| TCR beta chain FR4 of | SEQ ID NO: 30 | | |
| IMC-C103C | | | |
| TCR alpha chain of the | SEQ ID NO: 14 | SEQ ID NO: 40 | Referred to as |
| ImmTAC designated as | | | a13kaLQ |
| IMC-C103C | | | in WO 2017/ |
| | | | 175006 |
| TCR alpha chain | SEQ ID NO: 15 | — | |
| constant region of | | | |
| IMC-C103C | | | |
| TCR beta chain-anti- | SEQ ID NO: 16 | SEQ ID NO: 45 | Referred to as |
| CD3 portion of the | | | b21L in WO |
| ImmTAC designated | | | 2017/175006 |
| as IMC-C103C | | | |
| Anti-CD3 scFv portion | SEQ ID NO: 17 | — | Corresponds |
| of IMC-C103C | | | to amino |
| | | | acids |
| | | | 1-253 of |
| | | | SEQ ID NO: |
| | | | 16 of present |
| | | | application |
| Linker of IMC-C103C | SEQ ID NO: 18 | SEQ ID NO: 30 | |
| TCR beta chain | SEQ ID NO: 19 | — | |
| constant region of | | | |
| IMC-C103C | | | |
| Alternative linker | SEQ ID NO: 20 | SEQ ID NO: 31 | |
| sequence | | | |
| Alternative linker | SEQ ID NO: 21 | SEQ ID NO: 32 | |
| sequence | | | |
| Alternative linker | SEQ ID NO: 22 | SEQ ID NO: 33 | |
| sequence | | | |
| Alternative linker | SEQ ID NO: 23 | SEQ ID NO: 34 | |
| sequence | | | |
| Alternative linker | SEQ ID NO: 24 | SEQ ID NO: 35 | |
| sequence | | | |
| Alternative linker | SEQ ID NO: 25 | SEQ ID NO: 36 | |
| sequence | | | |
| Alternative linker | SEQ ID NO: 26 | SEQ ID NO: 37 | |
| sequence | | | |

The ImmTAC designated as IMC-C103C and which is described in the present application is designated ImmTAC4 in WO2017/175006.

In this aspect of the present invention, the TCR-anti-CD3 fusion molecule is administered as follows:
(a) at least one first dose in the range of from 10-20 μg;
(b) at least one second dose in the range of from 40-50 μg; and then
(c) at least one third dose in the range of from 90-400 μg,
wherein doses are administered every 6-8 days.

The dosage regimen of the present invention is a dose escalation regimen, in which increasing doses of the TCR-anti-CD3 fusion molecule are sequentially administered. Doses are thus administered in the specified order: first dose, then second dose, then third dose. By “first dose” is meant a dose of the TCR-anti-CD3 fusion molecule at a first level within the specified range. By “second dose” is meant a dose of the TCR-anti-CD3 fusion molecule at a second level within the specified range, which is higher than the first level. By “third dose” is meant a dose of the TCR-anti-CD3 fusion molecule at a third level within the specified range, which is higher than the second level. It will be appreciated that according to the dosage regimen of the present invention, a patient may receive more than three doses of the TCR-anti-CD3 fusion molecule, because more than one first dose, more than one second dose and/or more than one third dose may be administered.

In the present invention, the respective doses are expressed as a specified weight of therapeutic irrespective of the patient's weight or whether the same amount of therapeutic would be administered if calculated through one of the other methods routinely used to calculate an appropriate dosage for a patient, such as weight of therapeutic per kg of body weight, body surface area or lean muscle mass etc. The specified weight of therapeutic is typically administered at weekly intervals, e.g. on days 1, 8, 15, 22, etc of the treatment regimen, but the dosing interval could be longer or shorter. Thus, doses (each dose, i.e. at least one first dose, at least one second dose and at least one third dose) are administered every 6-8 days. Preferably, doses (i.e. at least one first dose, at least one second dose and at least one third dose) are administered every 7 days. The respective doses may be separated by different intervals. Alternatively, they may be separated by the same interval.

The first dose is in the range of from 10-20 μg. It may be in the range of from 11-19 μg, 12-18 μg, 13-17 μg or 14-16 μg. The dose may be 10, 11, 12, 13, 14, 15, 16, 17, 18, 19 or 20 μg. One preferred first dose is 15 μg, which may be administered on a weekly basis. One first dose may be administered. Alternatively, more than one first dose, for example 2-5 first doses, such as two first doses, may be administered. This may be required, for example, if the patient experiences an adverse event (AE) following administration of the first dose. In this case, it may be preferred to administer one or more additional first doses before escalating to the second dose. It is preferred that, if two or more first doses are administered, they are the same. However, they may be different.

The second dose is in the range of from 40-50 μg. It may be in the range of from 41-49 μg, 42-48 μg, 43-47 μg or 44-46 μg. The second dose may be 40, 41, 42, 43, 44, 45, 46, 47, 48, 49 or 50 μg. One preferred second dose is 45 μg, which may be administered on a weekly basis. One second dose may be administered. Alternatively, more than one second dose, for example 2-5 second doses, such as two second doses, may be administered. Again, this may be required, for example, if the patient experiences an adverse event following administration of the second dose. In this case, it may be preferred to administer one or more additional first doses before escalating to the second dose. It is preferred that, if two or more second doses are administered, they are the same. However, they may be different.

The third dose is in the range of from 90-400 μg. It may be in the range of from 100-380 μg, 110-350 μg, 120-330 μg, 130-300 μg, 140-280 μg, 150-260 μg, 160-240 μg, 180-230 μg or 200-220 μg. For example, it may be in the range of from 140-240 μg. The dose may be 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 μg. One preferred third dose is 140 μg. Another preferred third dose is 180 μg and a yet further is 240 μg. One or more third doses may be administered. Typically, multiple third doses are administered, for example, every 6-8 days, preferably every 7 days, until treatment is stopped. Treatment may be continued for one or more months or one or more years.

Treatment may be stopped, for example due to unacceptable toxicity or because the patient has shown an unacceptable level of disease progression. Alternatively, treatment may be stopped, for example, because the patient's symptoms have reduced in severity and/or tumour has shrunk to a level at which treatment with the TCR-anti-CD3 fusion molecule is deemed no longer necessary. The decision on whether and when to stop treatment can be determined by a clinician. The same dose may be used subsequently. Alternatively, the dose may be escalated. For example, the dose may be 5, 10, 15, 20, 30, 40 or 50 μg higher.

Any combination of ranges of first, second and third doses as described herein are contemplated. For example, the first dose may be in the range of from 13-17 μg or 14-16 μg, the second dose may be in the range of from 43-47 μg or 44-46 μg and the third dose may be in the range of from 120-300 μg or 140-250 μg, in any combination.

The first dose may be 15 μg, the second dose may be 45 μg and the third dose may be in the range of from 140-240 μg. The first dose may be 15 μg, the second dose may be 45 μg and the third dose may be 140 μg, 180 μg or 240 μg. In other words, the first dose may be 15 μg, the second dose may be 45 μg and the third dose may be 140 μg. The first dose may be 15 μg, the second dose may be 45 μg and the third dose may be 180 μg. The first dose may be 15 μg, the second dose may be 45 μg and the third dose may be 240 μg.

In a second aspect, the invention provides a TCR-anti-CD3 fusion molecule comprising:
a TCR alpha chain amino acid sequence of SEQ ID NO: 14 or a TCR alpha chain amino acid sequence that has at least 90% identity to the amino acid sequence of SEQ ID NO: 14, and
   a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
   wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
   for use in a method of treating MAGE-A4 positive cancer in a patient comprising administering the TCR-anti-CD3 fusion molecule to said patient intravenously, wherein the method comprises administration of:
   (a) at least one first dose;
   (b) at least one second dose; and then
   (c) at least one third dose in the range of from 90-400 μg,
   wherein the second dose is higher than the first dose and the third dose is higher than the second dose, and
   wherein doses are administered every 6-8 days.

This aspect of the invention relates to a dose escalation dosage regimen in which at least one third dose in the range of from 90-400 μg is administered following a dose escalation.

Doses (each dose, i.e. at least one first dose, at least one second dose and at least one third dose) are administered every 6-8 days. Preferably, doses (i.e. at least one first dose, at least one second dose and at least one third dose) are administered every 7 days. The respective doses may be separated by different intervals. Alternatively, they may be separated by the same interval.

The first and second dose may be determined by a clinician, or may be as defined herein in relation to the first aspect of the invention.

The third dose is in the range of from 90-400 μg, as described herein in relation to the first aspect of the invention. It may be in the range of from 100-380 μg, 110-350 μg, 120-330 μg, 130-300 μg, 140-280 μg, 150-260 μg, 160-240 μg, 180-230 μg or 200-220 μg. For example, it may be in the range of from 140-240 μg. The dose may be 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 μg. One preferred third dose is 140 μg. Another preferred third dose is 180 μg and a yet further is 240 μg. One or more third doses may be administered. Typically, multiple third doses are administered, for example, every 6-8 days, preferably every 7 days, until treatment is stopped. Treatment may be continued for one or more months or one or more years.

Treatment may be stopped, for example due to unacceptable toxicity or because the patient has shown an unacceptable level of disease progression. Alternatively, treatment may be stopped, for example, because the patient's symptoms have reduced in severity and/or tumour has shrunk to a level at which treatment with the TCR-anti-CD3 fusion molecule is deemed no longer necessary. The decision on whether and when to stop treatment can be determined by a clinician. The same dose may be used subsequently. Alternatively, the dose may be escalated. For example, the dose may be 5, 10, 15, 20, 30, 40 or 50 μg higher.

In the present invention, the TCR-anti-CD3 fusion molecule is administered intravenously (iv), typically by intravenous infusion.

The TCR-anti-CD3 fusion molecule for use in the present invention may be administered following a premedication regimen. As will be appreciated by a person of skill in the art, a premedication is the administration of medication prior to the administration of a treatment and is intended to counteract the potential side effects of the treatment.

For example, the TCR-anti-CD3 fusion molecule may be administered following a steroid (corticosteroid) and/or non-steroid based premedication regimen. The steroid may be administered prior to administering the first, second and/or third dose, and is typically administered prior to administering the third dose. The steroid may be administered, for example, 15, 20, 25 or 30 minutes prior to administering the first, second and/or third dose. The steroid may be administered when the third dose is 140 μg or above (for example, when the third dose is 150, 160, 170, 180, 190, 200, 210, 220, 230, 240, 250, 260, 270, 280, 290, 300, 310, 320, 330, 340, 350, 360, 370, 380, 390 or 400 μg) and/or only prior to the third dose being administered for the first time.

The steroid may be dexamethasone. Dexamethasone may be administered intravenously and may be given at a dose in the range of 4-6 mg. Higher doses may be used, for example 8, 12, or 20 mg. Alternative steroids include prednisone, methylprednisolone and hydrocortisone.

Other premedications for use in combination with the dosage regimen of the invention include:
Paracetamol-typically administered orally at 1 g or equivalent
Ibuprofen-typically administered orally at 600-800 mg or equivalent
Diphenhydramine-typically administered orally at 50 mg
Non-sedating antihistamines such as cetirizine (typically administered orally at 10 mg) or loratadine (typically administered orally at 10 mg)
An anti-emetic such as ondansetron (typically administered orally or intravenously at 8 mg)
Intravenous fluids—typically administered in the range of from 0.5-1 L. This is to mitigate the risk of hypotension, especially if the patient is experiencing dehydration, poor oral intake and/or nausea/vomiting

Any such premedications may be used alone or in combination. The premedication(s) may be administered prior to administering the first, second and/or third dose, typically prior to administering the third dose. The premedication(s) may be administered, for example, 15, 20, 25 or 30 minutes prior to administering the first, second and/or third dose. If the patient develops an adverse event associated with any of these premedications, a reduced dose may be given. For example, a dose of 25 mg of diphenhydramine may be administered.

The TCR-anti-CD3 fusion molecule for use in the present invention may be administered as a monotherapy. Alternatively, it may be administered in combination with one or more anti-cancer therapies, preferably immuno-modulatory therapies. Such therapies include:
checkpoint inhibitors such as agents that target PD-1 or PD-L1, e.g. atezolizumab (TECENTRIQ®), pembrolizumab, nivolumab, avelumab and durvalumab, and agents that target CTLA-4 such as ipilimumab and tremelimumab,
chemotherapy agents, such as dacarbazine and temozolamide,
immunotherapeutic agents, such as interleukin-2 (IL-2) and interferon (IFN)
BRAF inhibitors, such as vemurafenib and dabrafenib,
MEK inhibitors, such as trametinib,
TGF-β inhibitors such as galunisertib,
MET kinase inhibitors such as merestinib,
anti-angiogenic agents such as bevacizumab (Avastin®)

The TCR-anti-CD3 fusion molecule may be administered in combination with a checkpoint inhibitor. The checkpoint inhibitor may be atezolizumab. Atezolizumab may enhance the initial activity of IMC-C103C and sustain the effectiveness of the emerging anti-tumour immune response. Also, the combination therapy may aid in overcoming tumour resistance to PD-1/PD-L1 monotherapy by redirecting effector T cells into tumours, where poor T cell infiltration has been shown to be a key resistance mechanism to checkpoint inhibitors.

A preferred combination therapy uses atezolizumab in combination with a TCR-anti-CD3 fusion molecule as described herein. Atezolizumab is typically administered according to the current prescribing information, e.g. 840 mg every 2 weeks, 1200 mg every 3 weeks or 1680 mg every 4 weeks, by intravenous infusion.

The TCR-anti-CD3 fusion molecule may be administered in combination with other therapeutic agents sequentially. The TCR-anti-CD3 fusion molecule may be administered on its own for the first and subsequent doses, with the additional therapeutic agents being added thereafter, or vice-versa. In embodiments of the present invention where a TCR-anti-CD3 fusion molecule and another anti-cancer therapy are administered in combination, the TCR-anti-CD3 fusion molecule may be administered alone in weeks 1 and 2 and the other anti-cancer therapy added in week 3 and subsequent weeks. For example, atezolizumab is typically administered once the patient has reached the third dose of the TCR-anti-CD3 fusion molecule. Typically, atezolizumab is administered prior to administration of the TCR-anti-CD3 fusion molecule. Administration of the TCR-anti-CD3 fusion molecule, typically by intravenous infusion, may commence, for example, 30 mins after infusion of atezolizumab.

Combination therapies may lead to an increased risk of immune-related toxicities, such as CRS. Accordingly, the dose of a TCR-anti-CD3 fusion molecule may be initially given as a single agent prior to combination dosing. Dosing of one or more additional anti-cancer therapies may be administered from week 3.

The present invention relates to the treatment of MAGE-A4 positive cancers. By “MAGE-A4 positive cancer”, it is meant a cancer in which at least some of the cancer cells express MAGE-A4. MAGE-A4 expression can be assessed using any method known in the art, including, for example, histological methods. However, the invention is not intended to be limited to the treatment of cancers for which MAGE-A4 expression can be detected by histological methods.

MAGE-A4 positive cancers include, but are not limited to, ovarian cancer, lung cancer, head and neck cancer, oesophageal cancer, breast cancer, synovial sarcoma, gastric cancer, bladder cancer, and any tumour with squamous cell histology. The head and neck cancer may be head and neck squamous cell carcinoma (HNSCC). The lung cancer may be non-small cell lung carcinoma (NSCLC). The bladder cancer may be urothelial carcinoma. The oesophageal cancer may be gastroesophageal junction (GEJ) adenocarcinoma. The ovarian cancer may be epithelial ovarian cancer, such as high grade serous ovarian cancer.

The first aspect of the invention also extends to the use of a TCR anti-CD3 fusion molecule in the manufacture of a medicament for the treatment of MAGE-A4 positive cancer by intravenous administration of a TCR-anti-CD3 fusion molecule as defined herein, wherein the treatment of MAGE-A4 positive cancer comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days

The second aspect of the invention also extends to the use of a TCR anti-CD3 fusion molecule in the manufacture of a medicament for the treatment of MAGE-A4 positive cancer by intravenous administration of a TCR-anti-CD3 fusion molecule as defined herein, wherein the treatment of MAGE-A4 positive cancer comprises administration of:
(a) at least one first dose;
(b) at least one second dose; and then
(c) at least one third dose in the range of from 90-400 μg,
wherein the second dose is higher than the first dose and the third dose is higher than the second dose, and
wherein doses are administered every 6-8 days.

The first aspect of the invention also extends to a method of treating MAGE-A4 positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days.

The second aspect of the invention also extends to a method of treating MAGE-A4 positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose;
(b) at least one second dose; and then
(c) at least one third dose in the range of from 90-400 μg,
wherein the second dose is higher than the first dose and the third dose is higher than the second dose, and
wherein doses are administered every 6-8 days.

Methods of treating MAGE-A4 positive cancer include administering a therapeutically effective amount of a TCR anti-CD3 fusion molecule.

The TCR anti-CD3 fusion molecule can be formulated in pharmaceutical compositions. These compositions can comprise, in addition to the TCR anti-CD3 fusion molecule, one or more pharmaceutically acceptable excipients, carriers, buffers, stabilizers, or other materials well known to those skilled in the art. It may be provided in unit dosage form, will generally be provided in a sealed container and may be provided as part of a kit. Such a kit would normally (although not necessarily) include instructions for use and may include a plurality of said unit dosage forms.

The pharmaceutical composition may be in any suitable form for intravenous administration. Such compositions may be prepared by any method known in the art of pharmacy, for example by mixing the active ingredient with the carrier(s) or excipient(s) under sterile conditions.

Administration of the TCR anti-CD3 fusion molecule is preferably in a “therapeutically effective amount,” this being an amount sufficient to show benefit to the patient. In relation to the first aspect of the invention, the therapeutically effective amount comprises at least one first dose in the range of 10-20 μg, at least one second dose in the range of 40-50 μg, and at least one third dose in the range of 90-400 μg. In relation to the second aspect of the invention, the therapeutically effective amount comprises at least one first dose and/or at least one second dose as determined by a clinician taking account of the state of disease and the condition of the patient being treated. In relation to the second aspect of the invention, the therapeutically effective amount comprises at least one third dose in the range of 90-400 μg.

Administration of the TCR anti-CD3 fusion molecule to the patient may result in an improved outcome for the patient. For example, an increased duration of progression free survival or overall survival. Administration of the TCR anti-CD3 fusion molecule to the patient may result in decrease in overall tumor size as determined by RECIST v1.1 criteria (Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45(2):228-247).

Preferred features of each aspect of the invention are as for each of the other aspects mutatis mutandis. It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the TCR-anti-CD3 fusion molecule for use and method for treating MAGE-A4 positive cancer are specifically embraced by the present invention and are disclosed herein just as if each and every combination was individually and explicitly disclosed. The published documents mentioned herein are incorporated to the fullest extent permitted by law. Citation or identification of any document in this application is not an admission that such document is available as prior art to the present invention.

The specification is considered to be sufficient to enable one skilled in the art to practice the compositions and methods of the present disclosure. Various modifications in addition to those shown and described herein will become apparent to those skilled in the art from the foregoing description and fall within the scope of the appended claims. All publications, patents, and patent applications cited herein are hereby incorporated by reference in their entirety for all purposes.

The invention is further described in the following non-limiting Example.

### EXAMPLE

The present disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase 1/2 Study of TCR-Anti-CD3 Fusion Molecule in Humans

IMC-C103C-101 is a Phase 1/2 multicenter, open-label, first-in-human study of IMC-C103C as a monotherapy and in a combination with atezolizumab in human leukocyte antigen allele A*02:01 (HLA-A*02:01)-positive patients with melanoma-associated antigen-A4 (MAGE-A4) positive solid tumors including non-small cell lung cancer (NSCLC), esophageal carcinoma, gastric carcinoma, head and neck squamous cell carcinoma (HNSCC), urothelial carcinoma, ovarian carcinoma, or synovial sarcoma.

IMC-C103C is an immune-mobilizing monoclonal T cell receptor against cancer (ImmTAC®), a bispecific protein therapeutic comprising a soluble, affinity-enhanced T cell receptor (TCR; targeting domain) fused to an antibody single-chain variable fragment (scFv; effector domain). The IMC-C103C TCR recognizes a peptide from the tumor-associated antigen MAGE-A4, presented by HLA-A*02:01. Once the soluble TCR is engaged, the scFv effector can bind to CD3 on any T cell, redirecting the T cell to produce effector cytokines and/or kill the cell presenting the target peptide. In addition, IMC-C103C-mediated lysis may prime an endogenous anti-tumor immune response.

This example describes data from 44 patients treated in the dose escalation phase of monotherapy arm of IMC-C103C-101, across dose 10 cohorts.

IMC-C103C was administered via by weekly (Q1W) IV infusion in 21-day cycles. The duration of IV infusion was typically 1 hour±10 minutes in Cycles 1 and 2 and 30 minutes (±10 minutes) starting at Cycle 3 Day 1.

Safety assessments included physical examination, vital signs, weight, Eastern Cooperative Oncology Group (ECOG) performance status, hematology, chemistry, coagulation, urinalysis, thyroid function, cytokine testing, pregnancy testing, cardiac testing, as well as AE collection. Adverse Events (AEs) were graded according to National Cancer Institute Common Terminology Criteria for Adverse Events (NCI CTCAE) v5.0 unless otherwise specified.

Tumor response was determined locally according to RECIST v1.1.

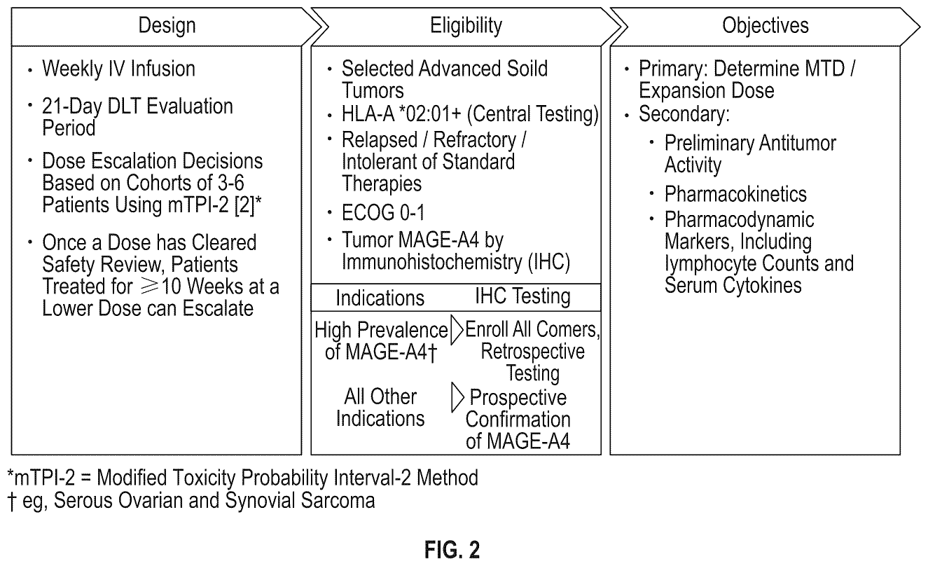

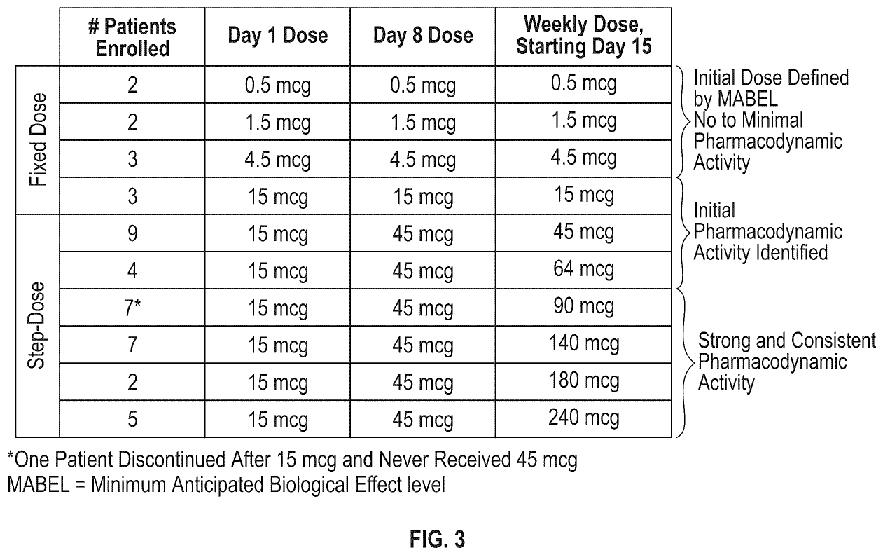

The design of the study is shown in FIG. 2. FIG. 3 shows the dose escalation schema.

Table 2 shows the baseline characteristics of participants in the study.

TABLE 2

| Characteristic | All doses, N = 44* |
|---|---|
| Age Mean (Range) | 59.5 (18, 83) yrs |
| Sex Female n (%) | 34 (77%) |
| ECOG performance status | |
| 0 n (%) | 20 (45%) |
| 1 n (%) | 24 (55%) |
| Indication | |
| Ovarian | 30 (68%) |
| Synovial sarcoma | 7 (16%) |
| NSCLC | 3 (7%) |
| HNSCC | 2 (5%) |
| Esophageal | 1 (2%) |
| Urothelial | 1 (2%) |
| MAGE-A4 H-Score Median (Range) | 12 (0, 300) |

*One patient enrolled at 90 mcg and 9 months after discontinuing study treatment was re-enrolled at 180 mcg.

Ovarian carcinoma was the most frequently enrolled indication. Patients were heavily pre-treated (median 4 prior lines). Patients were enrolled regardless of MAGE-A4 H-score; 27% were negative and 67% were positive by immunohistochemistry (IHC). Median MAGE-A4 IHC H-Score was very low for all comers (8) and for MAGE-A4+ (18).

The safety profile was found to be consistent with the mechanism of T cell activation. Table 3 shows adverse events (AEs) associated with administration of IMC-C103C.

The most common related AEs were consistent with CRS and generally dose dependent. They were typically Grade 1 or 2, occurring in first 3 weeks, and rapidly resolving. The most common related Grade 3 or 4 AE was neutropenia, which typically occurred at doses ≥90 mcg and was reversible (with treatment interruption or G-CSF) and not dose-limiting.

Three patients had related AEs that met dose-limiting toxicity (DLT) criteria:
1^{st} dose of 15 mcg (assigned to 15/45/90 mcg): Grade 3 worsening pleural effusion that resolved (patient received one additional 15 mcg dose and then had severe COVID and disease progression)
1^{st} dose of 240 mcg: Grade 3 AST increased that rapidly resolved (patient continued at 240 mcg with normal-Grade 1 AST until disease progression)
1^{st} dose of 240 mcg: Grade 3 CRS that resolved (patient currently on 140 mcg without CRS recurrence and with ongoing unconfirmed PR [−44%]) No related AE led to treatment discontinuation or death.

TABLE 3

| | | | | |
|---|---|---|---|---|
| | 0.5-4.5 | 15-64 | 90-240 | |
| | mcg | mcg | mcg | TOTAL |
| Preferred Term* | (n = 7) | (n = 16) | (n = 21) | (N = 44†) |

All Grades (treatment-related events in ≥20% of total patients)

| | | | | |
|---|---|---|---|---|
| Chills | — | 8 (50%) | 13 (62%) | 21 (48%) |
| Pyrexia* | 2 (29%) | 7 (44%) | 12 (57%) | 21 (48%) |
| Cytokine release syndrome‡ | 1 (14%) | 4 (25%) | 11 (52%) | 16 (36%) |
| Headache | 1 (14%) | 6 (38%) | 7 (33%) | 14 (32%) |
| Nausea | 1 (14%) | 6 (38%) | 6 (29%) | 13 (30%) |
| Hypotension* | — | 6 (38%) | 5 (24%) | 11 (25%) |
| Fatigue | 1 (14%) | 4 (25%) | 5 (24%) | 10 (23%) |

Grade 3-4 (treatment-related events in ≥5% of total patients)

| | | | | |
|---|---|---|---|---|
| Neutropenia/ Neutrophil count | — | 1 (6%) | 7 (33%) | 8 (18%) |
| decreased | | | | |
| Lymphocyte count decreased | 1 (14%) | 1 (6%) | 2 (10%) | 4 (9%) |
| ALT increased | — | 1 (6%) | 1 (5%) | 2 (5%) |
| AST increased | — | 1 (6%) | 1 (5%) | 2 (5%) |
| Headache | 1 | 1 (6%) | 1 (5%) | 2 (5%) |

*Includes events reported as a sign/symptom of CRS
†One patient enrolled at 90 mcg and 9 months after discontinuing study treatment was re-enrolled at 180 mcg
‡Cytokine release syndrome (CRS) was graded by the Investigators using ASTCT criteria (Lee DW, et al. ASTCT
Consensus Grading for Cytokine Release Syndrome and Neurologic Toxicity Associated with Immune Effector Cells. Biol Blood Marrow Transplant 2019; 25: 625-638). All other events were graded using NCI CTCAE v5.0.

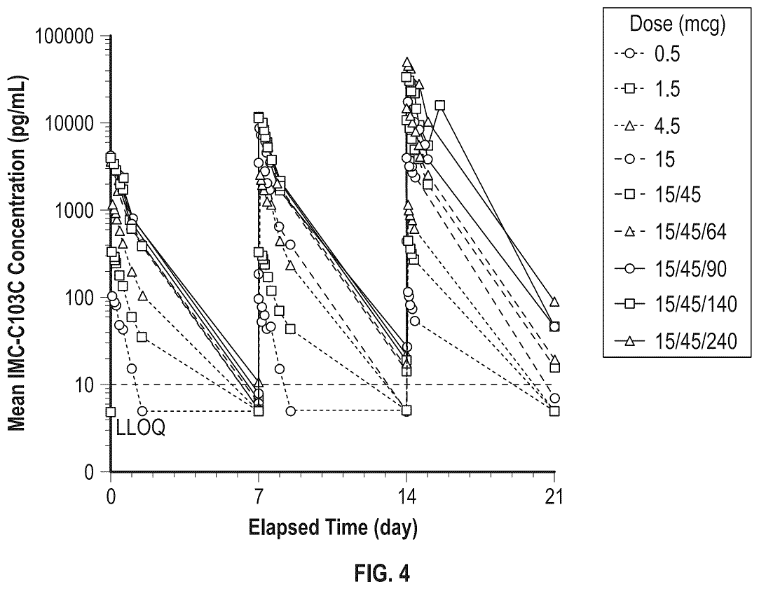

FIG. 4 shows the results of weekly IV dosing with 20 hr half-life in peripheral blood. PK data was available for the first 42 patients.

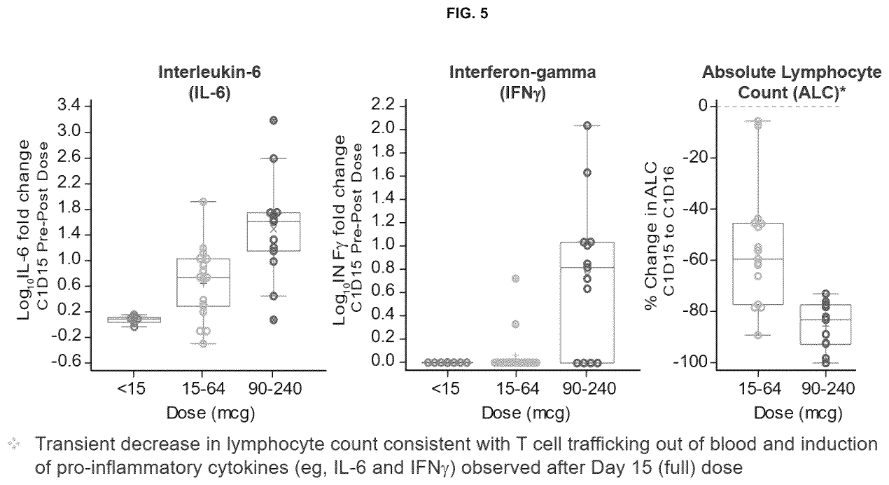

FIG. 5 shows consistent and robust evidence of T cell activity at ≥90 mcg IMC-C103C. A transient decrease in lymphocyte count consistent with T cell trafficking out of blood and induction of pro-inflammatory cytokines (eg, IL-6 and IFNγ) was observed after Day 15 (full) dose.

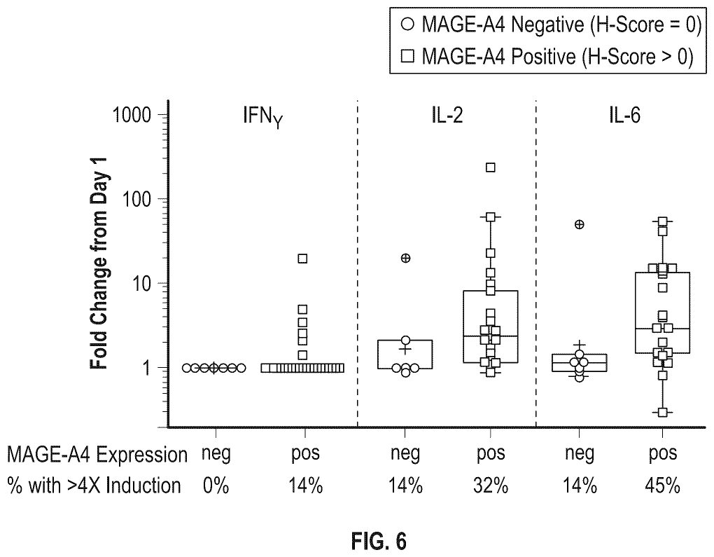

To evaluate the effect of tumor MAGE-A4 expression, serum cytokines were analyzed after 15 mcg on Day 1 (n=29). Results are shown in FIG. 6. IFNγ induction was only observed in patients with MAGE-A4 positive tumors. Median IL-2 and IL-6 induction were higher in patients with MAGE-A4 positive tumors.

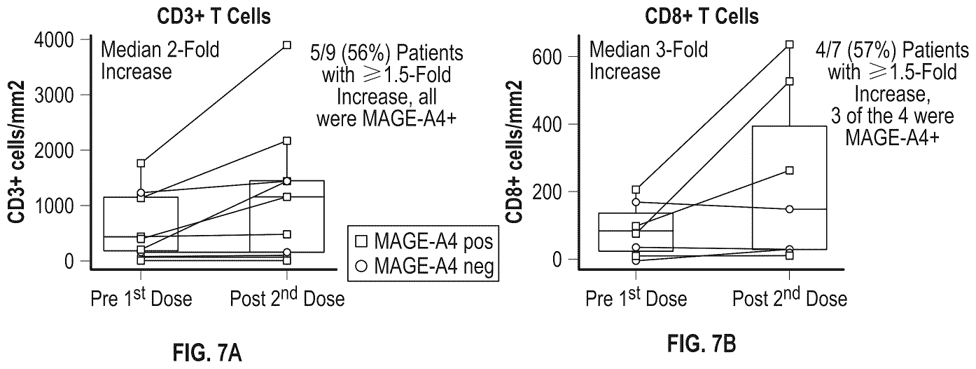

FIG. 7 shows increased T cell infiltration into MAGE-A4+ tumors. FIG. 7A shows infiltration of CD3+ T cells; 5/9 (56%) patients showed a ≥1.5-fold increase, all were MAGE-A4+. FIG. 7B shows infiltration of CD8+ T cells; 4/7 (57%) patients showed a ≥1.5-fold increase, 3 of the 4 were MAGE-A4+.

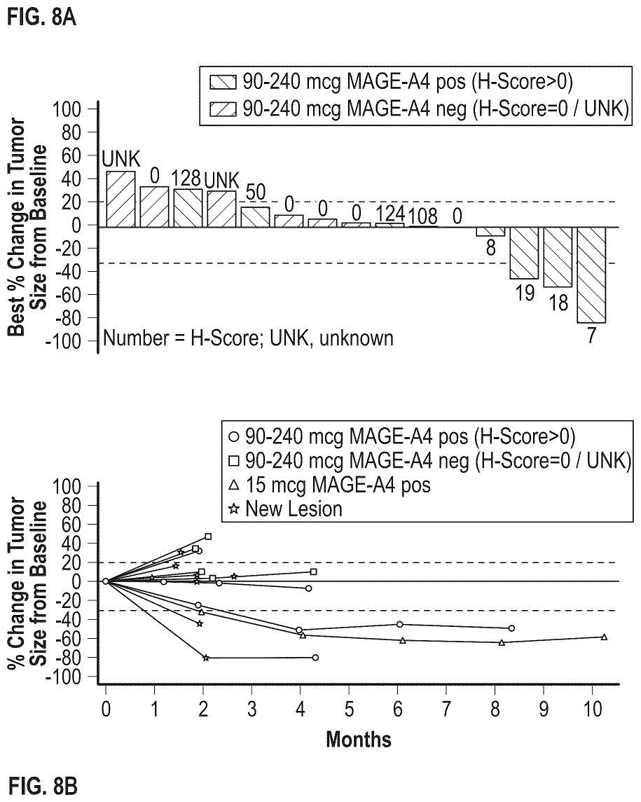

FIG. 8 demonstrates clinical activity of IMC-C103C in ovarian carcinoma at doses of 90-240 mcg. At <90 mcg, one ovarian cancer patient (H score=16) had durable partial response (PR). At 90-240 mcg, 17 ovarian patients were treated (15 had initial tumor assessment and 2 not yet evaluable). Of these 15 patients, 8 had MAGE-A4+ ovarian cancer (all had low H-Score <130). 4 of 8 patients with MAGE-A4+ tumors had tumor shrinkage, including one durable, confirmed PR.

TABLE 4
Clinical activity to date*

| Indication | H-Score | Dose | Response | DOR |
|---|---|---|---|---|
| Ovarian | 16 | 15 mcg | Confirmed PR | 8.3 mo |
| Ovarian | 18 | 90 mcg | Confirmed PR | 4.4+ mo |
| | | | (ongoing) | |
| Ovarian | 7 | 140 mcg | Overall TL | |
| | | | reduction (~81%) | |
| | | | but new lesions | |
| Ovarian | 19 | 140 mcg | Overall TL | |
| | | | reduction (~44%) | |
| | | | but new lesions | |
| HNSCC | 285 | 240 mcg | Unconfirmed PR | |
| | | | (ongoing) | |

*20 patients treated with 90-240 mcg (18 are efficacy evaluable, of whom 11 had MAGE-4A+ tumor by IHC) including 17 ovarian cancer patients (15 are efficacy evaluable; 8 with H score >0); 1 HNSCC (H score = 285); 1 patient with urothelial carcinoma (H score = 3); and 1 patient with esophageal carcinoma (H score = 175).

In 22 ctDNA evaluable patients across multiple dose levels, reduction in ctDNA was associated with longer overall survival (data not shown).

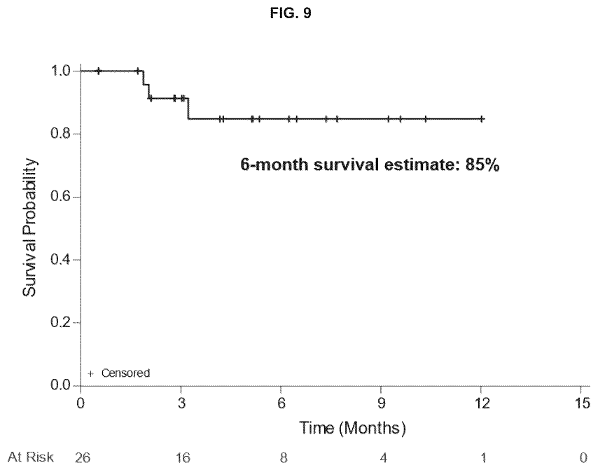

FIG. 9 shows overall survival in patients with ovarian carcinoma at doses ≥15 mcg. 26 ovarian carcinoma patients received ≥15 mcg. All had prior platinum (85% relapsed/refractory), 92% prior bevacizumab and all had low H-score (median=18). Although follow-up is ongoing, the 6-month OS rate ˜85%. Historically, the median OS for similar population is ˜10-11 months.

### CONCLUSIONS

IMC-C103C had a manageable safety profile in this study; the AEs were primarily cytokine-mediated. There were no treatment-related AEs leading to discontinuation or death. Consistent and robust biomarkers of T cell activation were observed at doses ≥90 mcg. Substantial increase in T cell infiltration in the tumor was observed. Durable partial responses were observed in ovarian carcinoma despite low MAGE-A4 expression and an unconfirmed partial response was observed in head and neck carcinoma.

The results demonstrate that IMC-C103C is well-tolerated in humans. A two step escalation regimen in which IMC-C103C is administered weekly by IV at a dose of 15 mcg on day 1, 45 mcg on day 8 and 160 mcg on day 15, and thereafter, is tolerable and is being further investigated an expansion phase in patients with ovarian carcinoma.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

What is claimed is:

1. A TCR-anti-CD3 fusion molecule comprising:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating MAGE-A4 positive cancer in a patient comprising administering the TCR-anti-CD3 fusion molecule to said patient intravenously, wherein the method comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days.

2. The TCR-anti CD3 fusion molecule for use according to claim 1, wherein the TCR-anti-CD3 fusion molecule comprises an alpha chain amino acid sequence corresponding to SEQ ID NO: 14 and a TCR beta chain-anti-CD3 amino acid sequence corresponding to SEQ ID NO: 16.

3. The TCR-anti-CD3 fusion molecule for use according to claim 1 or 2, wherein the first dose is 15 μg, the second dose is 45 μg and the third dose is in the range of from 140 μg-240 μg.

4. The TCR-anti-CD3 fusion molecule for use according to claim 3, wherein the third dose is 140 μg, 180 μg or 240 μg.

5. The TCR-anti-CD3 fusion molecule for use according to claim 1 or 2, wherein the first dose is 15 μg, the second dose is 45 μg, and the third dose is 90 μg, 140 μg, 180 μg, or 240 μg.

6. The TCR-anti-CD3 fusion molecule for use according to any preceding claim, wherein a further third dose is administered every 6-8 days until treatment is stopped.

7. The TCR-anti-CD3 fusion molecule for use according to any preceding claim, wherein a steroid is administered prior to the first, second and/or third dose.

8. The TCR-anti-CD3 fusion molecule for use according to any preceding claim, which is administered in combination with one or more anti-cancer therapies.

9. The TCR-anti-CD3 fusion molecule for use according to claim 8, wherein the anti-cancer therapy is a checkpoint inhibitor.

10. The TCR-anti-CD3 fusion molecule for use according to claim 9, wherein the checkpoint inhibitor is atezolizumab.

11. The TCR-anti-CD3 fusion molecule for use according to any preceding claim, wherein the MAGE-A4 positive cancer is selected from the group consisting of ovarian cancer, lung cancer, head and neck cancer, oesophageal cancer, breast cancer, synovial sarcoma, gastric cancer, bladder cancer and a tumour with squamous cell histology.

12. A method of treating MAGE-A4 positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose in the range of from 10-20 μg;
(b) at least one second dose in the range of from 40-50 μg; and then
(c) at least one third dose in the range of from 90-400 μg,
wherein doses are administered every 6-8 days.


SEQUENCE LISTING (30 sequences)

SEQ ID NO: 1 - protein, 10 aa, synthetic construct
GVYDGREHTV

SEQ ID NO: 2 - protein, 113 aa, synthetic construct
ANQVEQSPQSLIILEGKNVTLQCNYTVSPFSNLRWYKQDTGRGPVSLTILTFSENTKSNGRYTATLDADTKQSSLHITASQLSDSASYICVVNSAQGLYIPTFGRGTSLIVHP

SEQ ID NO: 3 - protein, 6 aa, synthetic construct
VSPFSN

SEQ ID NO: 4 - protein, 7 aa, synthetic construct
LTFSENT

SEQ ID NO: 5 - protein, 13 aa, synthetic construct
VVNSAQGLYIPTF

SEQ ID NO: 6 - protein, 17 aa, synthetic construct
LRWYKQDTGRGPVSLTI

SEQ ID NO: 7 - protein, 34 aa, synthetic construct
KSNGRYTATLDADTKQSSLHITASQLSDSASYIC

SEQ ID NO: 8 - protein, 116 aa, synthetic construct
DVKVTQSSRYLVKRTGEKVFLECVQDLDHENMFWYRQDPGLGLRLIYFSRFATGKEKGDIPEGYSVSREKKERFSLILESASTNQTSMYLCASSSDQNSGDPYEQYFGPGTRLTVT

SEQ ID NO: 9 - protein, 5 aa, synthetic construct
LDHEN

SEQ ID NO: 10 - protein, 6 aa, synthetic construct
SRFATG

SEQ ID NO: 11 - protein, 16 aa, synthetic construct
ASSSDQNSGDPYEQYF

SEQ ID NO: 12 - protein, 17 aa, synthetic construct
MFWYRQDPGLGLRLIYF

SEQ ID NO: 13 - protein, 37 aa, synthetic construct
KEKGDIPEGYSVSREKKERFSLILESASTNQTSMYLC

SEQ ID NO: 14 - protein, 199 aa, synthetic construct
ANQVEQSPQSLIILEGKNVTLQCNYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 15 - protein, 86 aa, synthetic construct
YIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 16 - protein, 504 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDVKVTQSSRYLVKRTGEKVFLECVQDLDHENMFWYRQDPGLGLRLIYFSRFATGKEKGDIPEGYSVSREKKERFSLILESASTNQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 17 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 18 - protein, 5 aa, synthetic construct
GGGGS

SEQ ID NO: 19 - protein, 130 aa, synthetic construct
EDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 20 - protein, 5 aa, synthetic construct
GGGSG

SEQ ID NO: 21 - protein, 5 aa, synthetic construct
GGSGG

SEQ ID NO: 22 - protein, 5 aa, synthetic construct
GSGGG

SEQ ID NO: 23 - protein, 6 aa, synthetic construct
GSGGGP

SEQ ID NO: 24 - protein, 5 aa, synthetic construct
GGEPS

SEQ ID NO: 25 - protein, 7 aa, synthetic construct
GGEGGGP

SEQ ID NO: 26 - protein, 12 aa, synthetic construct
GGEGGGSEGGGS

SEQ ID NO: 27 - protein, 26 aa, synthetic construct
ANQVEQSPQSLIILEGKNVTLQCNYT

SEQ ID NO: 28 - protein, 10 aa, synthetic construct
GRGTSLIVHP

SEQ ID NO: 29 - protein, 26 aa, synthetic construct
DVKVTQSSRYLVKRTGEKVFLECVQD

SEQ ID NO: 30 - protein, 9 aa, synthetic construct
GPGTRLTVT